(19) United States
(12) Patent Application Publication
(10) Pub. No.: US 2012/0121653 A1
(43) Pub. Date: May 17, 2012

(54) Novel mometasone compositions and methods of making and using the same

(75) Inventors: Scott Jenkins, Downingtown, PA (US); Gary G. Liversidge, West Chester, PA (US)

(73) Assignee: Elan Pharma International Limited

(21) Appl. No.: 11/633,088

(22) Filed: Dec. 4, 2006

Related U.S. Application Data
(60) Provisional application No. 60/741,452, filed on Dec. 2, 2005.

(51) Int. Cl. A61K 31/58; A61P 29/00; A61P 11/06; A61K 9/14; A61P 11/00; B82Y 5/00

(52) U.S. Cl. 424/400; 514/172; 977/773; 977/788; 977/906; 977/915

(57) ABSTRACT

The present invention is directed to mometasone furoate compositions comprising mometasone furoate and at least one surface stabilizer. The mometasone furoate particles of the composition preferably have an effective average particle size of less than about 2000 nm.

### CROSS-REFERENCE TO RELATED PATENT APPLICATIONS

This application claims the benefit under 35 U.S.C. §119(e) to U.S. Provisional Application No. 60/741,452, filed on Dec. 2, 2005, which is incorporated herein in its entirety by reference and using such compositions.

### FIELD OF THE INVENTION

The present invention relates to a composition comprising mometasone furoate and at least one surface stabilizer, and methods of making and using such compositions.

### BACKGROUND OF THE INVENTION

### A. Background Regarding Nanoparticulate Compositions

Nanoparticulate compositions, first described in U.S. Pat. No. 5,145,684 (“the '684 patent”), are particles comprising a poorly soluble therapeutic or diagnostic agent having associated with the surface thereof a non-crosslinked surface stabilizer. The '684 patent does not describe nanoparticulate compositions of mometasone furoate.

Methods of making nanoparticulate compositions are described, for example, in U.S. Patent Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for

“Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for “Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(-)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive nanoparticulate compositions having cationic surface stabilizers;” U.S. Pat. No. 6,431,478 for “Small Scale Mill;” U.S. Pat. No. 6,432,381 for “Methods for Targeting Drug Delivery to the Upper and/or Lower Gastrointestinal Tract,” U.S. Pat. No. 6,582,285 for “Apparatus for Sanitary Wet Milling,” U.S. Pat. No. 6,592,903 for “Nanoparticulate Dispersions Comprising a Synergistic Combination of a Polymeric Surface Stabilizer and Dioctyl Sodium Sulfosuccinate,” U.S. Pat. No. 6,656,504 for “Nanoparticulate Compositions Comprising Amorphous Cyclosporine and Methods of Making and Using Such Compositions,” U.S. Pat. No. 6,582,285 for “Apparatus for Sanitary Wet Milling;” U.S. Pat. No. 6,592,903 for “Nanoparticulate Dispersions Comprising a Synergistic Combination of a Polymeric Surface Stabilizer and Dioctyl Sodium Sulfosuccinate,” U.S. Pat. No. 6,742,734 for “System and Method for Milling Materials,” U.S. Pat. No. 6,745,962 for “Small Scale Mill and Method Thereof,” U.S. Pat. No. 6,811,767 for “Liquid droplet aerosols of nanoparticulate drugs,” U.S. Pat. No. 6,908,626 for “Compositions having a combination of immediate release and controlled release characteristics,” U.S. Pat. No. 6,969,529 for “Nanoparticulate compositions comprising copolymers of vinyl pyrrolidone and vinyl acetate as surface stabilizers,” U.S. Pat. No. 6,976,647 for “System and Method for Milling Materials,” U.S. Pat. No. 6,991,191 for “Method of Using a Small Scale Mill,” U.S. Pat. No. 7,101,576 for “Nanoparticulate Megestrol Formulation,” all of which are specifically incorporated by reference. None of these references describe nanoparticulate compositions of mometasone furoate.

In addition, U.S. Patent Publication No. 20060246142 for “Nanoparticulate quinazoline derivative formulations,” U.S. Patent Publication No. 20060246141 for “Nanoparticulate lipase inhibitor formulations,” U.S. Patent Publication No. 20060216353 for “Nanoparticulate corticosteroid and antihistamine formulations,” U.S. Patent Publication No. 20060210639 for “Nanoparticulate bisphosphonate compositions,” U.S. Patent Publication No. 20060210638 for “Injectable compositions of nanoparticulate immunosuppressive compounds,” U.S. Patent Publication No. 20060204588 for “Formulations of a nanoparticulate finasteride, dutasteride or tamsulosin hydrochloride, and mixtures thereof,” U.S. Patent Publication No. 20060198896 for “Aerosol and injectable formulations of nanoparticulate benzodiazepine,” U.S. Patent Publication No. 20060193920 for “Nanoparticulate Compositions of Mitogen-Activated (MAP) Kinase Inhibitors,” U.S. Patent Publication No. 20060188566 for “Nanoparticulate formulations of docetaxel and analogues thereof,” U.S. Patent Publication No. 20060165806 for “Nanoparticulate candesartan formulations,” “U.S. Patent Publication No. 20060159767 for “Nanoparticulate bicalutamide formulations,” U.S. Patent Publication No. 20060159766 for “Nanoparticulate tacrolimus formulations,” U.S. Patent Publication No. 20060159628 for “Nanoparticulate benzothiophene formulations,” U.S. Patent Publication No. 20060154918 for “Injectable nanoparticulate olanzapine formulations,” U.S. Patent Publication No. 20060121112 for “Topiramate pharmaceutical composition,” U.S. Patent Publication No. 20020012675 Al, for “Controlled Release Nanoparticulate Compositions,” U.S. Patent Publication No. 20040195413 Al, for “Compositions and method for milling materials,” U.S. Patent Publication No. 20040173696 Al for “Milling microgram quantities of nanoparticulate candidate compounds,” U.S. Patent Publication No. 20050276974 for “Nanoparticulate Fibrate Formulations;” U.S. Patent Publication No. 20050238725 for “Nanoparticulate Compositions Having a Peptide as a Surface Stabilizer;” U.S. Patent Publication No. 20050233001 for “Nanoparticulate Megestrol Formulations;” U.S. Patent Publication No. 20050147664 for “Compositions Comprising Antibodies and Methods of Using the Same for Targeting Nanoparticulate Active Agent Delivery;” U.S. Patent Publication No. 20050063913 for “Novel Metaxalone Compositions;” U.S. Patent Publication No. 20050042177 for “Novel Compositions of Sildenafil Free Base;” U.S. Patent Publication No. 20050031691 for “Gel Stabilized Nanoparticulate Active Agent Compositions;” U.S. Patent Publication No. 20050019412 for “Novel Glipizide Compositions;” U.S. Patent Publication No. 20050004049 for “Novel Griseofulvin Compositions;” U.S. Patent Publication No. 20040258758 for “Nanoparticulate Topiramate Formulations;” U.S. Patent Publication No. 20040258757 for “ Liquid Dosage Compositions of Stable Nanoparticulate Active Agents;” U.S. Patent Publication No. 20040229038 for “Nanoparticulate Meloxicam Formulations;” U.S. Patent Publication No. 20040208833 for “Novel Fluticasone Formulations;” U.S. Patent Publication No. 20040156895 for “Solid Dosage Forms Comprising Pullulan;” U.S. Patent Publication No. 20040156872 for “Novel Nimesulide Compositions;” U.S. Patent Publication No. 20040141925 for “Novel Triamcinolone Compositions;” U.S. Patent Publication No. 20040115134 for “Novel Nifedipine Compositions;” U.S. Patent Publication No. 20040105889 for “Low Viscosity Liquid Dosage Forms;” U.S. Patent

Publication No. 20040105778 for “Gamma Irradiation of Solid Nanoparticulate Active Agents;” U.S. Patent Publication No. 20040101566 for “Novel Benzoyl Peroxide Compositions;” U.S. Patent Publication No. 20040057905 for “Nanoparticulate Beclomethasone Dipropionate Compositions;” U.S. Patent Publication No. 20040033267 for “Nanoparticulate Compositions of Angiogenesis Inhibitors;” U.S. Patent Publication No. 20040033202 for “Nanoparticulate Sterol Formulations and Novel Sterol Combinations;” U.S. Patent Publication No. 20040018242 for “Nanoparticulate Nystatin Formulations;” U.S. Patent Publication No. 20040015134 for “Drug Delivery Systems and Methods;” U.S. Patent Publication No. 20030232796 for “Nanoparticulate Polycosanol Formulations & Novel Polycosanol Combinations;” U.S. Patent Publication No. 20030215502 for “Fast Dissolving Dosage Forms Having Reduced Friability;” U.S. Patent Publication No. 20030185869 for “Nanoparticulate Compositions Having Lysozyme as a Surface Stabilizer;” U.S. Patent Publication No. 20030181411 for “Nanoparticulate Compositions of Mitogen-Activated Protein (MAP) Kinase Inhibitors;” U.S. Patent Publication No. 20030137067 for “Compositions Having a Combination of Immediate Release and Controlled Release Characteristics;” U.S. Patent Publication No. 20030108616 for “Nanoparticulate Compositions Comprising Copolymers of Vinyl Pyrrolidone and Vinyl Acetate as Surface Stabilizers;” U.S. Patent Publication No. 20030095928 for “Nanoparticulate Insulin;” U.S. Patent Publication No. 20030087308 for “Method for High Through- put Screening Using a Small Scale Mill or Microfluidics;” U.S. Patent Publication No. 20030023203 for “Drug Delivery Systems & Methods;” U.S. Patent Publication No. 20020179758 for “System and Method for Milling Materials;” and U.S. Patent Publication No. 20010053664 for “Apparatus for Sanitary Wet Milling,” describe nanoparticulate active agent compositions and are specifically incorporated by reference. None of these references describe compositions of nanoparticulate mometasone furoate.

Amorphous small particle compositions are described, for example, in U.S. Patent No. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter.”

### B. Background Regarding Mometasone Furoate

Mometasone furoate is a synthetic anti-inflammatory corticosteroid having the chemical name of 9,21-Dichloro-11, β,17-di-hydroxy-16^{α}-methylpregna-1,4-diene-3 ,20-done 17-(2 furoate). Mometasone furoate has the empirical formula C₂₇H₃₀Cl₂O₆ and a molecular weight of 521.45. Likewise, mometasone furoate monohydrate has the empirical formula C₂₇H₃₀Cl₂O₆ H₂O and a molecular weight of 539.45 and is a white powder.

Mometasone furoate monohydrate is practically insoluble in water, slightly soluble in methanol, ethanol, and isopropanol; soluble in acetone and chloroform, and freely soluble in tetrahydrofuran. Its partition coefficient between octanol and water is greater than 5000.

Mometasone furoate is described and claimed in U.S. Patent Nos. 5,837,699; 6,127,353; and 6,723,713; all to Schering Corporation. The compound has anti-inflammatory activity and is particularly useful for the treatment of respiratory disorders, particularly upper airway diseases.

Depending on the mode of administration, mometasone furoate can be used to treat, for example, corticosteroid-responsive diseases of the upper and lower airway passages and lungs, such as seasonal (e.g., hay fever) or perennial rhinitis, which are characterized by seasonal or perennial sneezing, rhinorrhea, nasal congestion, pruritis and eye itching, redness and tearing, and nonallergic (vasomotor) rhinitis (i.e., eosinophilic nonallergic rhinitis which is found in patients with negative skin tests and those who have numerous eosinophils in their nasal secretions). The term “allergic rhinitis” as used herein includes any allergic reaction of the nasal mucosa.

In addition, the mometasone furoate compositions described herein can be used to treat asthma, including any asthmatic condition marked by recurrent attacks of paroxysmal dyspnea (i.e., reversible obstructive airway passage disease) with wheezing due to spasmodic contraction of the bronchi, Asthmatic conditions which may be treated or prevented in accordance with this invention include allergic asthma and bronchial allergy characterized by manifestations in sensitized persons provoked by a variety of factors including exercise, especially vigorous exercise (exercise induced bronchospasm), irritant particles (e.g., pollen, dust, cotton, dander, etc.), as well as mild to moderate asthma, chronic asthma, severe chronic asthma, severe and unstable asthma, nocturnal asthma, and psychological stresses.

Mometasone furoate is also approved for topical dermatologic use to treat inflammatory and/or pruritic manifestations of corticosteroid-responsive dermatoses. Thus, like other topical corticosteroids, mometasone furoate has anti-inflammatory, antipruritic, and vasoconstrictive properties

Mometasone furoate is marketed as NASONEX® Nasal Spray (Shering Corporation) and mometasone furoate monohydrate is the active component in this commercial product. NASONEX® Nasal Spray, 50 mcg is a metered-dose, manual pump spray unit containing an aqueous suspension of mometasone furoate monohydrate equivalent to 0.05% w/w mometasone furoate calculated on the anhydrous basis; in an aqueous medium containing glycerin, microcrystalline cellulose and carboxymethylcellulose, sodium citrate, 0.25% w/w phenylethyl alcohol, citric acid, benzalkonium chloride, and polysorbate 80, The pH is between 4.3 and 4.9.

After initial priming (10 actuations), each actuation of the pump delivers a metered spray containing 100 mg of suspension containing mometasone furoate monohydrate equivalent to 50 mcg of mometasone furoate calculated on the anhydrous basis. NASONEX is a corticosteroid and the precise mechanism of corticosteroid action on allergic rhinitis is not known. Corticosteroids have been shown to have a wide range of effects on multiple cell types (e.g., mast cells, eosinophils, neutrophils, macrophages, and lymphocytes) and mediators (e.g., histamine, eicosanoids, leukotrienes, and cytokines) involved in inflammation. Intranasal corticosteroids may cause a reduction in growth velocity when administered to pediatric patients.

Adverse reactions from the current marketed form of mometasone furoate monohydrate include headache, viral infection, pharyngitis, eptistaxis/blood-tinged mucus, coughing, upper respiratory tract infection, dysmenorrheal, musculoskeletal pain, sinusitis and vomiting.

There are several disadvantages with conventional nasal dosage forms of mometasone furoate monohydrate, including the use of benzalkonium chloride as a preservative. The presence of benzalkonium chloride limits the use of these formulations because some patients are allergic to benzalkonium chloride and other patients find the smell to be unpleasant.

Delivery of drugs to the nasal mucosa can also be accomplished with aqueous, propellant-based, or dry powder formulations. However, absorption of poorly soluble drugs can be problematic because of mucociliary clearance which transports deposited particles from the nasal mucosa to the throat where they are swallowed. Complete clearance generally occurs within about 15-20 minutes. Thus, poorly soluble drugs which do not dissolve within this time frame are unavailable for either local or systemic activity.

The development of aerosol drug delivery systems has been hampered by the inherent instability of aerosols, the difficulty of formulating dry powder and aqueous aerosols of water-insoluble drugs, and the difficulty of designing an optimal drug particle size for an aerosol drug delivery system. Thus, there is a need in the art for aerosols that deliver an optimal dosage of essentially insoluble drugs throughout the respiratory tract or nasal cavity. The present invention satisfies these needs.

### SUMMARY OF THE INVENTION

The present invention relates to compositions comprising mometasone furoate and at least one surface stabilizer. The mometasone furoate particles in the composition may have an effective average particle size of less than about 2000 nm.

Another aspect of the invention is directed to pharmaceutical compositions comprising a mometasone furoate composition of the invention. The pharmaceutical compositions preferably comprise mometasone furoate, at least one surface stabilizer, and at least one pharmaceutically acceptable carrier, as well as any desired excipients.

Moreover, the invention is directed to mometasone furoate compositions which can be sterile filtered.

In yet another embodiment, the invention is directed to bioadhesive mometasone furoate formulations. Such compositions are useful, for example, for oral, nasal, or topical applications.” In a preferred embodiment, the mometasone furoate compositions of the present invention are formulated for nasal application.

This invention further discloses a method of making a mometasone furoate composition. Such a method comprises contacting mometasone furoate and at least one surface stabilizer for a time and under conditions sufficient to provide a mometasone furoate composition in which the mometasone furoate particles have an effective average particle size of less than about 2 microns. The one or more surface stabilizers can be contacted with mometasone furoate either before, during, or after size reduction of the mometasone furoate .

Finally, the invention is directed to methods of treatment using the mometasone furoate compositions of the invention.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### DETAILED DESCRIPTION OF THE INVENTION

The present invention is directed to compositions comprising mometasone furoate and at least one surface stabilizer. The mometasone furoate particles in the composition may have an effective average particle size of less than about 2000 nm.

As taught in the '684 patent, not every combination of surface stabilizer and active agent will result in a stable nanoparticulate composition. It was surprisingly discovered that stable nanoparticulate mometasone furoate formulations can be made.

The current formulations of mometasone furoate for oral, nasal, or topical administration suffer from the following problems: (1) the poor solubility of the drug necessitates making a suspension in water or a dry powder for oral or nasal administration; (2) conventional formulations often contain benzalkonium chloride as a preservative, which may cause allergic reactions in some patients; (3) poor bioavailability; and (4) a variety of side effects are associated with the current mometasone furoate dosage forms.

A nanoparticulate mlation of mometasone furoate monohydrate can be sterile filtered, thereby eliminating the need for benzalkonium chloride as a preservative.

Moreover, a nanoparticulate formulation of mometasone furoate would be more efficacious than the conventional formulation since the nanoparticulate active agent would provide better surface coverage at the same dose. This would, in turn, lead to a faster onset of effect.

The present invention overcomes problems encountered with the prior art mometasone furoate formulations. Specifically, the mometasone furoate compositions of the invention may offer the following advantages: (1) the composition can be formulated in a dried form which readily redisperses; (2) the composition may offer a potential decrease in the frequency of dosing; (3) smaller doses of drug may be required to obtain the same pharmacological effect as compared to conventional microcrystalline or soluble forms of mometasone furoate; (4) bioadhesive mometasone furoate compositions that can coat the nasal or pulmonary cavity, or the desired site of application for dermatological applications and be retained for a period of time, thereby increasing the efficacy of the drug as well as eliminating or decreasing the frequency of dosing; (5) nanoparticulate mometasone furoate formulations having very small particle sizes can be sterile filtered; and (6) the nanoparticulate mometasone furoate compositions of the invention do not require organic solvents or pH extremes.

The present invention is described herein using several definitions, as set forth below and throughout the application.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein with reference to stable drug particles, “stable” includes, but is not limited to, one or more of the following parameters: (1) that the mometasone furoate particles do not appreciably flocculate or agglomerate due to interparticle attractive forces, or otherwise significantly increase in particle size over time; (2) that the physical structure of the mometasone furoate particles is not altered over time, such as by conversion from an amorphous phase to crystalline phase; (3) that the mometasone furoate particles are chemically stable; and/or (4) where the mometasone furoate has not been subject to a heating step at or above the melting point of the mometasone furoate in the preparation of the nanoparticles of the invention.

“Conventional active agents or drugs” refers to non-nanoparticulate compositions of active agents or solubilized active agents or drugs. Non-nanoparticulate active agents have an effective average particle size of greater than about 2 microns, meaning that at least 50% of the active agent particles have a size greater than about 2 microns. (Nanoparticulate active agents as defined herein have an effective average particle size of less than about 2 microns. Further, the nanoparticulate active agent refers to multiple forms of the active agent, including amorphous and crystalline forms.

“Pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salts” as used herein refers to derivatives wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

“Therapeutically effective amount” as used herein with respect to a drug dosage, shall mean that dosage that provides the specific pharmacological response for which the drug is administered in a significant number of subjects in need of such treatment. It is emphasized that ‘therapeutically effective amount,’ administered to a particular subject in a particular instance will not always be effective in treating the diseases described herein, even though such dosage is deemed a “therapeutically effective amount” by those skilled in the art. Therapeutically effective amount also includes an amount that is effective for prophylaxis. It is to be further understood that drug dosages are, in particular instances, measured as oral dosages, or with reference to drug levels as measured in blood.

### C. Preferred Characteristics of the Nanoparticulate Mometasone Furoate Compositions of the Invention

1. Lower Doses and Frequency of Dosing Offered by the Mometasone Furoate Compositions of the Invention

The mometasone furoate compositions of the invention can be administered less frequently and at lower doses than the currently marketed forms of mometasone furoate. Lower dosages can be used because the small particle size of the mometasone furoate particles ensure greater absorption, and in the case of bioadhesive nanoparticulate mometasone furoate compositions, the mometasone furoate is retained at the desired site of application for a longer period of time as compared to conventional mometasone furoate dosage forms, thereby increasing the effectiveness of the dosage form. In one embodiment, the compositions of the present invention comprise a nanoparticulate formulation of mometasone furoate monohydrate in crystalline form.

2. Increased Bioavailability

The compositions of the invention comprising a nanoparticulate mometasone furoate, or a salt or derivative thereof, are proposed to exhibit increased bioavailability, and require smaller doses as compared to prior or conventional mometasone furoate formulations.

In some embodiments, the nanoparticulate mometasone furoate compositions, upon administration to a mammal, produce therapeutic results at a dosage which is less than that of a non-nanoparticulate dosage form of the same mometasone furoate. In addition, the need for a smaller dosage may decrease or eliminate the severity, intensity or duration of side effects associated with conventional non-nanoparticulate mometasone furoate compositions.

3. Bioadhesive Mometasone Furoate Compositions

The invention is also directed to bioadhesive mometasone furoate formulations for any suitable method of administration, such as but not limited to oral, nasal, or topical application. Bioadhesive formulations of the invention are primarily useful in nasal applications. Bioadhesive nanoparticulate compositions were first described in U.S. Pat. No. 6,428,814 for “Bioadhesive Nanoparticulate Compositions Having Cationic Surface Stabilizers.”

Bioadhesive mometasone furoate compositions comprise mometasone furoate particles and at least one surface stabilizer. The surface stabilizer may be an anionic surface stabilizer, a cationic surface stabilizer, a zwitterionic surface stabilizer, an ionic surface stabilizer, or a combination thereof. The mometasone furoate particles can have an effective average particle size of less than about 2 microns. The composition may also comprise one or more secondary surface stabilizers, which can be non-cationic.

Bioadhesive formulations of mometasone furoate exhibit exceptional bioadhesion to biological surfaces, such as hair, mucous, skin, etc. The term bioadhesion refers to any attractive interaction between two biological surfaces or between a biological and a synthetic surface. In the case of bioadhesive mometasone furoate compositions of the invention, the term bioadhesion is used to describe the adhesion between the mometasone furoate compositions and a biological substrate (i.e. gastrointestinal mucin, lung tissue, nasal mucosa, skin, etc.). There are basically two mechanisms which may be responsible for this bioadhesion phenomena: mechanical or physical interactions and chemical interactions. The first of these, mechanical or physical mechanisms, involves the physical interlocking or interpenetration between a bioadhesive entity and the receptor tissue, resulting from a good wetting of the bioadhesive surface, swelling of the bioadhesive polymer, penetration of the bioadhesive entity into a crevice of the tissue surface, or interpenetration of bioadhesive composition chains with those of the mucous or other such related tissues. The second possible mechanism of bioadhesion, chemical, incorporates strong primary bonds (i.e., covalent bonds) as well as weaker secondary forces such as ionic attraction, van der Waals interactions, and hydrogen bonds. It is believed that this chemical form of bioadhesion is primarily responsible for the bioadhesive properties of the mometasone furoate compositions described herein. However, physical and mechanical interactions may also play a secondary role in the bioadhesion of such mometasone furoate compositions.

Because of the character of biological surfaces, the surface stabilizers of the invention result in bioadhesive formulations. Surprisingly, the bioadhesive property of nanoparticulate active agent compositions comprising surface stabilizers diminishes as the particle size of the active agent increases, as noted in U.S. Pat. No. 6,428,814. The surface stabilizer may be an anionic surface stabilizer, a cationic surface stabilizer, a zwitterionic surface stabilizer, an ionic surface stabilizer, or a combination thereof.

The bioadhesive mometasone furoate compositions are useful in any situation in which it is desirable to apply the compositions to a biological surface. The bioadhesive mometasone furoate compositions of the invention coat the targeted surface in a continuous and uniform film which is invisible to the naked human eye.

The adhesion exhibited by the inventive compositions means that mometasone furoate particles are not easily washed off, rubbed off, or otherwise removed from the biological surface for an extended period of time. The period of time in which a biological cell surface is replaced is the factor that limits retention of the bioadhesive mometasone furoate particles to that biological surface. For example, skin cells are replaced every 24-48 hours. Thus, the mometasone furoate composition would have to be reapplied to the skin every 48 hours. Mucous cells shed and are replaced about every 5-6 hours. Other biological surfaces, such as chitin, hair, teeth, and bone, do not routinely shed cells and, therefore, repeat applications may not be necessary.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), polyvinylpyrrolidone-2-dimethylaminoethyl methacrylate dimethyl sulfate, 1,2 Dipalmitoyl-sn-Glycero-3-Phosphoethanolamine-N-[Amino(Polyethylene Glycol)2000] (sodium salt) (also known as DPPE-PEG(2000)-Amine Na) (Avanti Polar Lipids, Alabaster, Ala.), Poly(2-methacryloxyethyl trimethylammonium bromide) (Polysciences, Inc., Warrington, PA) (also known as S1001), poloxamines such as Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.), lysozyme, long-chain polymers such as alginic acid, carrageenan (FMC Corp.), and POLYOX (Dow, Midland, Mich.).

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₇₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar. In one embodiment, the mometasone furoate monohydrate formulation does not comprise benzalkonium chloride.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric cationic surface stabilizers are any nonpolymeric compound, such as benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄⁽⁺⁾:

(i) none of R₁-R₄ are CH₃;

(ii) one of R₁-R₄ is CH₃;

(iii) three of R₁-R₄ are CH₃;

(iv) all of R₁-R₄ are CH₃;

(v) two of R₁-R₄ are CH₃, one of R₁-R₄ is C₆H₅CH₂, and one of R₁-R₄ is an alkyl chain of seven carbon atoms or less;

(vi) two of R₁-R₄ are CH₃, one of R₁-R₄ is C₆H₅CH₂, and one of R₁-R₄ is an alkyl chain of nineteen carbon atoms or more;

(vii) two of R₁-R₄ are CH₃ and one of R₁-R₄ is the group C₆H₅(CH₂)ₙ, where n>1;

(viii) two of R₁-R₄ are CH₃, one of R₁-R₄ is C₆H₅CH₂, and one of R₁-R₄ comprises at least one heteroatom;

(ix) two of R₁-R₄ are CH₃, one of R₁-R₄ is C₆H₅CH₂, and one of R₁-R₄ comprises at least one halogen;

(x) two of R₁-R₄ are CH₃, one of R₁-R₄ is C₆H₅CH₂, and one of R₁-R₄ comprises at least one cyclic fragment;

(xi) two of R₁-R₄ are CH₃ and one of R₁-R₄ is a phenyl ring; or

(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quatemium-15), distearyldimonium chloride (Quaternium-5), dodecyl dimethyl ethylbenzyl ammonium chloride(Quatemium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquatemium-1, pro cainehydro chloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide. In one embodiment, the mometasone furoate monohydrate formulation does not comprise benzalkonium chloride.

Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated by reference.

### 4. Sterile Filterable Nanoparticulate Mometasone Furoate Compositions

According to the invention, a sterile filtered mometasone furoate composition may comprise: (1) mometasone furoate particles having an effective average particle size of less than about 200 nm, and (2) at least one surface stabilizer. Two or more surface stabilizers may be used in combination.

In other embodiments of the invention, the sterile filterable nanoparticulate mometasone furoate compositions have an effective average particle size of less than about 140 nm, less than about 130 nm, less than about 120 nm, less than about 110 nm, less than about 100 nm, less than about 90 nm, less than about 80 nm, less than about 70 nm, less than about 60 nm, or less than about 50 nm. Because the compositions have such a small effective average particle size, they can be readily sterile filtered.

In preferred embodiments of the invention, at least about 99.9% of the mometasone furoate particles have an effective average particle size of less than 200 nm (D99), or at least about 90% of the mometasone furoate particles having an effective average particle size of less than 130 nm (D90).

Filtration is a highly cost-effective method for sterilizing homogeneous solutions when the membrane filter pore size is less than or equal to about 0.2 microns (200 nm) because a 0.2 micron filter is sufficient to remove essentially all bacteria. Sterile filtration, in addition to being cost effective, has other advantages in that certain compounds are not able to be sterilized by other methods, such as heat or gamma irradiation.

Sterile filtration is normally not used to sterilize conventional suspensions of micron-sized drug particles because the drug particles are too large to pass through the membrane pores. In principle, 0.2 μm filtration can be used to sterilize nanoparticulate active agent compositions. However, because nanoparticulate active agent compositions have a size range, many of the particles of a typical nanoparticulate active agent composition having an average particle size of 200 nm may have a size greater than 200 nm. Such larger particles tend to clog the sterile filter. Thus, only nanoparticulate active agent compositions having very small average particle sizes can be sterile filtered.

The U.S. Food and Drug Administration has recently issued guidelines requiring aqueous orally inhaled products to be sterile. This is problematic for aerosol formulations of nanoparticulate drugs, as heat sterilization can result in crystal growth and particle aggregation of such formulations, and sterile filtration can be difficult because of the required small particle size of the composition.

Administration by inhalation of corticosteroids, compared with oral administration, is preferable as this mode of administration reduces the risk of systemic side effects. The reduced risk of side effect arises from the mode of administration because corticosteroids are highly active topically and only weakly active systemically, thereby minimizing effects on the pituitary-adrenal axis, the skin, and the eye. Side effects associated with inhalation therapy are primarily oropharyngeal candidiasis and dysphonia (due to atrophy of laryngeal muscles). Oral corticosteroids cause atrophy of the dermis with thin skin, striae, and ecchymoses but inhaled corticosteroids do not cause similar changes in the respiratory tract.

Other advantages of inhaled over oral administration include direct deposition of steroid in the airways which generally provides more predictable administration. The oral doses required for adequate control vary substantially, whereas inhaled corticosteroids are usually effective within a narrower range. There are, however, a number of factors that influence the availability of inhaled corticosteroids: extent of airway inflammation, degree of lung metabolism, amount of drug swallowed and metabolized in the gastrointestinal tract, the patient's ability to coordinate the release and inspiration of the medication, type of corticosteroid, and the delivery system.

A sterile inhaled mometasone furoate dosage form is particularly useful in treating immunocompromised patients, infants or juvenile patients, and the elderly, as these patient groups are the most susceptible to infection caused by a non-sterile mometasone furoate dosage form.

### 5. Low Viscosity Liquid Dosage Forms

A liquid dosage form of a conventional microcrystalline or non-nanoparticulate mometasone furoate composition would be expected to be a relatively large volume, highly viscous substance which would not be well accepted by patient populations. Moreover, viscous solutions can be problematic in parenteral and aerosol administration because these solutions require a slow syringe push and can stick to tubing. In addition, conventional formulations of poorly water-soluble active agents, such as mometasone furoate, tend to be unsafe for intravenous administration techniques, which are used primarily in conjunction with highly water-soluble substances.

Liquid dosage forms of the nanoparticulate mometasone furoate compositions of the invention provide significant advantages over a liquid dosage form of a conventional mometasone furoate microcrystalline compound. The low viscosity and silky texture of liquid dosage forms of the nanoparticulate mometasone furoate compositions of the invention result in advantages in both preparation and use. These advantages include, for example: (1) better subject compliance due to the perception of a lighter formulation which is easier to consume and digest; (2) ease of dispensing because one can use a cup or a syringe; (3) potential for formulating a higher concentration of mometasone furoate resulting in a smaller dosage volume and thus less volume for the subject to consume; and (4) easier overall formulation concerns.

Liquid mometasone furoate dosage forms which are easier to consume are especially important when considering juvenile patients, terminally ill patients, and elderly patients. Viscous or gritty formulations, and those that require a relatively large dosage volume, are not well tolerated by these patient populations. Liquid oral dosage forms can be particularly preferably for patient populations who have difficulty consuming tablets, such as infants and the elderly.

The viscosities of liquid dosage forms of nanoparticulate mometasone furoate according to the invention are preferably less than about 1/200, less than about 1/175, less than about 1/150, less than about 1/125, less than about 1/100, less than about 1/75, less than about 1/50, or less than about 1/25 of a liquid oral dosage form of a conventional, non-nanoparticulate mometasone furoate composition, at about the same concentration per ml of mometasone furoate.

Typically the viscosity of liquid nanoparticulate mometasone furoate dosage forms of the invention, at a shear rate of 0.1 (l/s), is from about 2000 mPa s to about 1 mPa s, from about 1900 mPa·s to about 1 mPa·s, from about 1800 mPa·s to about 1 mPa·s, from about 1700 mPa·s to about 1 mPa·s, from about 1600 mPa·s to about 1 mPa·s, from about 1500 mPa·s to about 1 mPa·s, from about 1400 mPa·s to about 1 mPa·s, from about 1300 mPa·s to about 1 mPa·s, from about 1200 mPa·s to about 1 mPa·s, from about 1100 mPa·s to about 1 mPa·s, from about 1000 mPa·s to about 1 mPa·s, from about 900 mPa·s to about 1 mPa·s, from about 800 mPa·s to about 1 mPa·s, from about 700 mPa·s to about 1 mPa·s, from about 600 mPa·s to about 1 mPa·s, from about 500 mPa·s to about 1 mPa·s, from about 400 mPa·s to about 1 mPa·s, from about 300 mPa·s to about 1 mPa·s, from about 200 mPa·s to about 1 mPa·s, from about 175 mPa·s to about 1 mPa·s, from about 150 mPa·s to about 1 mPa·s, from about 125 mPa·s to about 1 mPa·s, from about 100 mPa·s to about 1 mPa·s, from about 75 mPa·s to about 1 mPa·s, from about 50 mPa·s to about 1 mPa·s, from about 25 mPa·s to about 1 mPa·s, from about 15 mPa·s to about 1 mPa·s, from about 10 mPa·s to about 1 mPa·s, or from about 5 mPa·s to about 1 mPa·s. Such a viscosity is much more attractive for subject consumption and may lead to better overall subject compliance.

Viscosity is concentration and temperature dependent. Typically, a higher concentration results in a higher viscosity, while a higher temperature results in a lower viscosity. Viscosity as defined above refers to measurements taken at about 20° C. (The viscosity of water at 20° C. is 1 mPa s.) The invention encompasses equivalent viscosities measured at different temperatures.

Another important aspect of the invention is that the nanoparticulate mometasone furoate compositions of the invention are not turbid. “Turbid,” as used herein refers to the property of particulate matter that can be seen with the naked eye or that which can be felt as “gritty.” The nanoparticulate mometasone furoate compositions of the invention can be poured out of or extracted from a container as easily as water, whereas a liquid dosage form of a non-nanoparticulate or solubilized mometasone furoate is expected to exhibit notably more “sluggish” characteristics.

The liquid formulations of this invention can be formulated for dosages in any volume but preferably equivalent or smaller volumes than a liquid dosage form of a conventional non-nanoparticulate mometasone furoate composition.

### 6. Redispersibility Profiles of Solid Dose Forms of the Nanoparticulate Mometasone Furoate Compositions of the Invention

An additional feature of solid dose forms of the nanoparticulate mometasone furoate compositions of the invention, such as dry powder aerosols, is that the dosage forms redisperse such that the effective average particle size of the redispersed mometasone furoate particles is less than about 2 microns. This is significant, as if upon administration the nanoparticulate mometasone furoate particles present in the compositions of the invention did not redisperse to a substantially nanoparticulate particle size, then the dosage form may lose the benefits afforded by formulating mometasone furoate into a nanoparticulate particle size.

This is because nanoparticulate mometasone furoate compositions benefit from the small particle size of mometasone furoate ; if the nanoparticulate mometasone furoate particles do not redisperse into the small particle sizes upon administration, then “clumps” or agglomerated mometasone furoate particles are formed. With the formation of such agglomerated particles, the bioavailability of the dosage form may fall.

Moreover, solid dose forms of the nanoparticulate mometasone furoate compositions of the invention exhibit dramatic redispersion of the nanoparticulate mometasone furoate particles upon administration to a mammal, such as a human or animal, as demonstrated by reconstitution in a biorelevant aqueous media. Such biorelevant aqueous media can be any aqueous media that exhibit the desired ionic strength and pH, which form the basis for the biorelevance of the media. The desired pH and ionic strength are those that are representative of physiological conditions found in the human body. Such biorelevant aqueous media can be, for example, aqueous electrolyte solutions or aqueous solutions of any salt, acid, or base, or a combination thereof, which exhibit the desired pH and ionic strength.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1 M while fasted state intestinal fluid has an ionic strength of about 0.14. See e.g., Lindahl et al., “Characterization of Fluids from the Stomach and Proximal Jejunum in Men and Women,” Pharm. Res., 14 (4): 497-502 (1997).

It is believed that the pH and ionic strength of the test solution is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 M, and NaCl solutions, ranging in concentration from about 0.001 to about 0.1 M, and mixtures thereof. For example, electrolyte solutions can be, but are not limited to, about 0.1 M HCl or less, about 0.01 M HCl or less, about 0.001 M HCl or less, about 0.1 M NaCl or less, about 0.01 M NaCl or less, about 0.001 M NaCl or less, and mixtures thereof. Of these electrolyte solutions, 0.01 M HCl and/or 0.1 M NaCl, are most representative of fasted human physiological conditions, owing to the pH and ionic strength conditions of the proximal gastrointestinal tract.

Electrolyte concentrations of 0.001 M HC1, 0.01 M HC1, and 0.1 M HCl correspond to pH 3, pH 2, and pH 1, respectively. Thus, a 0.01 M HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In other embodiments of the invention, the redispersed mometasone furoate particles of the invention (redispersed in an aqueous, biorelevant, or any other suitable media) have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

Redispersibility can be tested using any suitable means known in the art. See e.g., the example sections of U.S. Pat. No. 6,375,986 for “Solid Dose Nanoparticulate Compositions Comprising a Synergistic Combination of a Polymeric Surface Stabilizer and Dioctyl Sodium Sulfosuccinate.”

### 6. Improved Pharmacokinetic Profiles of the Mometasone Furoate Compositions

The nanoparticulate mometasone furoate compositions described herein may also exhibit a desirable pharmacokinetic profile when administered to mammalian subjects. The desirable pharmacokinetic profile of the mometasone furoate compositions preferably includes, but is not limited to: (1) a Cₘₐₓ for mometasone furoate or a derivative or salt thereof, when assayed in the plasma of a mammalian subject following administration, that is preferably greater than the Cₘₐₓ for a non-nanoparticulate formulation of the same mometasone furoate, administered at the same dosage; and/or (2) an AUC for mometasone furoate or a derivative or a salt thereof, when assayed in the plasma of a mammalian subject following administration, that is preferably greater than the AUC for a non-nanoparticulate formulation of the same mometasone furoate, administered at the same dosage; and/or (3) a Tₘₐₓ for mometasone furoate or a derivative or a salt thereof, when assayed in the plasma of a mammalian subject following administration, that is preferably less than the Tₘₐₓ for a non-nanoparticulate formulation of the same mometasone furoate, administered at the same dosage. The desirable pharmacokinetic profile, as used herein, is the pharmacokinetic profile measured after the initial dose of mometasone furoate or derivative or a salt thereof

In one embodiment, a composition comprising at least one nanoparticulate mometasone furoate or a derivative or salt thereof exhibits in comparative pharmacokinetic testing with a non-nanoparticulate formulation of the same mometasone furoate, administered at the same dosage, a Tₙₐₓ not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, or not greater than about 5% of the Tₘₐₓ exhibited by the non-nanoparticulate mometasone furoate formulation.

In another embodiment, the composition comprising at least one nanoparticulate mometasone furoate or a derivative or salt thereof, exhibits in comparative pharmacokinetic testing with a non-nanoparticulate formulation of the same mometasone furoate, administered at the same dosage, a Cₘₐₓ which is at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by the non-nanoparticulate mometasone furoate formulation.

In yet another embodiment, the composition comprising at least one nanoparticulate mometasone furoate or a derivative or salt thereof, exhibits in comparative pharmacokinetic testing with a non-nanoparticulate formulation of the same mometasone furoate, administered at the same dosage, an AUC which is at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate mometasone furoate formulation.

### 7. The Pharmacokinetic Profiles of the Mometasone Furoate Compositions are Not Affected by the Fed or Fasted State of the Subject Ingesting the Compositions

In some embodiments, the pharmacokinetic profile of the nanoparticulate mometasone furoate compositions are not substantially affected by the fed or fasted state of a subject ingesting the composition. This means that there would be little or no appreciable difference in the quantity of drug absorbed or the rate of drug absorption when the nanoparticulate mometasone furoate compositions are administered in the fed or fasted state.

Benefits of a dosage form which substantially eliminates the effect of food include an increase in subject convenience, thereby increasing subject compliance, as the subject does not need to ensure that they are taking a dose either with or without food. This is significant, as with poor subject compliance an increase in the medical condition for which the drug is being prescribed may be observed.

8. Bioequivalency of Mometasone Furoate Compositions When Administered in the Fed Versus the Fasted State

In some embodiments, administration of a nanoparticulate mometasone furoate composition to a subject in a fasted state is bioequivalent to administration of the composition to a subject in a fed state. The difference in absorption of the nanoparticulate mometasone furoate compositions, when administered in the fed versus the fasted state, preferably is less than about 100%, less than about 90%, less than about 80%, less than about 70%, less than about 60%, less than about 55%, less than about 50%, less than about 45%, less than about 40%, less than about 35%, less than about 30%, less than about 25%, less than about 20%, less than about 15%, less than about 10%, less than about 5%, or less than about 3%.

In some embodiments, the invention encompasses compositions comprising at least one nanoparticulate mometasone furoate, wherein administration of the composition to a subject in a fasted state is bioequivalent to administration of the composition to a subject in a fed state, in particular as defined by _{Cmax} and AUC guidelines given by the U.S. Food and Drug Administration and the corresponding European regulatory agency (EMEA). Under U.S. FDA guidelines, two products or methods are bioequivalent if the 90% Confidence Intervals (CI) for AUC and Cₘₐₓ are between 0.80 to 1.25 (Tₘₐₓ measurements are not relevant to bioequivalence for regulatory purposes). To show bioequivalency between two compounds or administration conditions pursuant to Europe's EMEA guidelines, the 90% CI for AUC must be between 0.80 to 1.25 and the 90% CI for Cₘₐₓ must between 0.70 to 1.43.

### 9. Dissolution Profiles of the Mometasone Furoate Compositions

The nanoparticulate mometasone furoate compositions are proposed to have unexpectedly dramatic dissolution profiles. Rapid dissolution of an administered active agent is preferable, as faster dissolution generally leads to faster onset of action and greater bioavailability. Additionally, a faster dissolution rate would allow for a larger dose of the drug to be absorbed, which would increase drug efficacy. To improve the dissolution profile and bioavailability of the mometasone furoate, it would be useful to increase the drug's dissolution so that it could attain a level close to 100%.

The mometasone furoate compositions of the invention preferably have a dissolution profile in which within about 5 minutes at least about 20% of the composition is dissolved. In other embodiments, at least about 30% or at least about 40% of the mometasone furoate composition is dissolved within about 5 minutes. In yet other embodiments, preferably at least about 40%, at least about 50%, at least about 60%, at least about 70%, or at least about 80% of the mometasone furoate composition is dissolved within about 10 minutes. In further embodiments, preferably at least about 70%, at least about 80%, at least about 90%, or at least about 100% of the mometasone furoate composition is dissolved within 20 minutes.

In some embodiments, dissolution is preferably measured in a medium which is discriminating. Such a dissolution medium will produce two very different dissolution curves for two products having very different dissolution profiles in gastric juices; i.e., the dissolution medium is predictive of in vivo dissolution of a composition. An exemplary dissolution medium is an aqueous medium containing the surfactant sodium lauryl sulfate at 0.025 M. Determination of the amount dissolved can be carried out by spectrophotometry. The rotating blade method (European Pharmacopoeia) can be used to measure dissolution.

### 10. Combination Active Agent Compositions

The invention encompasses the nanoparticulate mometasone furoate compositions of the invention formulated or co-administered with one or more non-mometasone furoate active agents, which are either conventional (solubilized or microparticulate) or nanoparticulate. Methods of using such combination compositions are also encompassed by the invention. The non- mometasone furoate active agents can be present in a crystalline phase, an amorphous phase, a semi-crystalline phase, a semi-amorphous phase, or a mixture thereof.

The compound to be administered in combination with a nanoparticulate mometasone furoate composition of the invention can be formulated separately from the nanoparticulate mometasone furoate composition or co-formulated with the nanoparticulate mometasone furoate composition. Where a nanoparticulate mometasone furoate composition is co-formulated with a second active agent, the second active agent can be formulated in any suitable manner, such as immediate-release, rapid-onset, sustained-release, or dual-release form.

If the non- mometasone furoate active agent has a nanoparticulate particle size i.e., a particle size of less than about 2 microns, then preferably it will have one or more surface stabilizers associated with the surface of the active agent. In addition, if the active agent has a nanoparticulate particle size, then it is preferably poorly soluble and dispersible in at least one liquid dispersion media. By “poorly soluble” it is meant that the active agent has a solubility in a liquid dispersion media of less than about 30 mg/mL, less than about 20 mg/mL, less than about 10 mg/mL, or less than about 1 mg/mL. Useful liquid dispersion medias include, but are not limited to, water, aqueous salt solutions, safflower oil, and solvents such as ethanol, t-butanol, hexane, and glycol.

Such non-mometasone furoate active agents can be, for example, a therapeutic agent. A therapeutic agent can be a pharmaceutical agent, including biologics. The active agent can be selected from a variety of known classes of drugs, including, for example, amino acids, proteins, peptides, nucleotides, anti-obesity drugs, central nervous system stimulants, carotenoids, corticosteroids, elastase inhibitors, anti-fungals, oncology therapies, anti-emetics, analgesics, cardiovascular agents, anti-inflammatory agents, such as NSAIDs and COX-2 inhibitors, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytics, sedatives (hypnotics and neuroleptics), astringents, alpha-adrenergic receptor blocking agents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators, and xanthines.

A description of these classes of active agents and a listing of species within each class can be found in Martindale's The Extra Pharmacopoeia, 31^{st} Edition (The Pharmaceutical Press, London, 1996), specifically incorporated by reference. The active agents are commercially available and/or can be prepared by techniques known in the art.

Exemplary nutraceuticals and dietary supplements are disclosed, for example, in Roberts et al., Nutraceuticals: The Complete Encyclopedia of Supplements, Herbs, Vitamins, and Healing Foods (American Nutraceutical Association, 2001), which is specifically incorporated by reference. Dietary supplements and nutraceuticals are also disclosed in Physicians' Desk Reference for Nutritional Supplements, 1st Ed. (2001) and The Physicians' Desk Reference for Herbal Medicines, 1st Ed. (2001), both of which are also incorporated by reference. A nutraceutical or dietary supplement, also known as a phytochemical or functional food, is generally any one of a class of dietary supplements, vitamins, minerals, herbs, or healing foods that have medical or pharmaceutical effects on the body.

Exemplary nutraceuticals or dietary supplements include, but are not limited to, lutein, folic acid, fatty acids (e.g., DHA and ARA), fruit and vegetable extracts, vitamin and mineral supplements, phosphatidylserine, lipoic acid, melatonin, glucosamine/chondroitin, Aloe Vera, Guggul, glutamine, amino acids (e.g., arginine, iso-leucine, leucine, lysine, methionine, phenylanine, threonine, tryptophan, and valine), green tea, lycopene, whole foods, food additives, herbs, phytonutrients, antioxidants, flavonoid constituents of fruits, evening primrose oil, flax seeds, fish and marine animal oils, and probiotics. Nutraceuticals and dietary supplements also include bio-engineered foods genetically engineered to have a desired property, also known as “pharmafoods.”

### D. Mometasone Furoate Compositions

The invention provides compositions comprising mometasone furoate particles and at least one surface stabilizer. The surface stabilizers adsorb to or associate with the surface of the mometasone furoate particles. Surface stabilizers useful herein do not chemically react with the mometasone furoate particles or itself. Individually adsorbed molecules of the surface stabilizer are essentially free of intermolecular cross-linkages. The compositions can comprise two or more surface stabilizers.

The present invention also includes mometasone furoate compositions together with one or more non-toxic physiologically acceptable carriers, adjuvants, or vehicles, collectively referred to as carriers.

The mometasone furoate compositions can be formulated for for administration via any suitable method, such as parenteral injection (e.g., intravenous, intramuscular, or subcutaneous), oral administration (in solid, liquid, or aerosol (i.e., pulmonary) form), vaginal, nasal, rectal, ocular, local (powders, creams, ointments or drops), buccal, intracisternal, intraperitoneal, topical administration, and the like. Exemplary mometasone furoate dosage forms of the invention include, but are not limited to, liquid dispersions, gels, powders, sprays, solid re-dispersable dosage forms, ointments, creams, aerosols (pulmonary and nasal), solid dose forms, etc. In other embodiments of the invention, the mometasone furoate compositions can be formulated: (a) for administration selected from the group consisting of parenteral, oral, pulmonary, intravenous, rectal, ophthalmic, colonic, intracisternal, intravaginal, intraperitoneal, ocular, otic, local, buccal, nasal, bioadhesive and topical administration; (b) into a dosage form selected from the group consisting of liquid dispersions, gels, aerosols, ointments, creams, lyophilized formulations, tablets, capsules; (c) into a dosage form selected from the group consisting of controlled release formulations, fast melt formulations, delayed release formulations, extended release formulations, pulsatile release formulations, mixed immediate release formulations, controlled release formulations; or (d) any combination of (a), (b), and (c).

1. Mometasone Furoate Particles

As used herein the term mometasone furoate refers to a synthetic anti-inflammatory corticosteroid having the chemical name of 9,21-Dichloro-11β,17-di-hydroxy-16^{α}-methylpregna-1,4-diene-3,20-done 17-(2 furoate) and salts and derivatives thereof. “Mometasone furoate” as used in this invention encompasses mometasone furoate monohydrate, as well as other forms of mometasone furoate.

Mometasone furoate has the empirical formula C₂₇H₃₀Cl₂O₆ and a molecular weight of 521.45. Likewise, mometasone furoate monohydrate has the empirical formula C₂₇H₃₀Cl₂O₆ H₂O and a molecular weight of 539.45 and is a white powder. Mometasone furoate monohydrate is practically insoluble in water, slightly soluble in methanol, ethanol, and isopropanol; soluble in acetone and chloroform, and freely soluble in tetrahydrofuran.

The mometasone furoate of the invention can be in a crystalline phase, an amorphous phase, a semi-crystalline phase, a semi-amorphous phase, or a mixture thereof. In a preferred embodiment, the mometasone furoate is in a crystalline form.

Mometasone furoate has anti-inflammatory activity and is particularly useful for the treatment of nasal symptoms of seasonal allergic and perennial allergic rhinitis.

2. Surface Stabilizers

Combinations of more than one surface stabilizer can be used in the invention. Useful surface stabilizers which can be employed in the invention include, but are not limited to, known organic and inorganic pharmaceutical excipients, such as ionic, non-ionic, anionic, cationic, and zwitterionic surfactants or compounds. Such excipients include various polymers, low molecular weight oligomers, natural products, and surfactants. Preferred surface stabilizers include non-ionic surfactants such as tyloxapol.

Depending upon the desired method of administration, bioadhesive formulations of mometasone furoate can be prepared by selecting one or more surface stabilizers that impart bioadhesive properties to the resultant composition. Exemplary surface stabilizers are also described above in Section A.3. The surface stabilizer may be an anionic surface stabilizer, a cationic surface stabilizer, a zwitterionic surface stabilizer, an ionic surface stabilizer, a non-ionic surface stabilizer, or any combination thereof.

Representative examples of other useful surface stabilizers include albumin (from any suitable species, including but not limited to human serum albumin and bovine serum albumin), hypromellose (previously known as hydroxypropyl methylcellulose or HPMC), hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxs 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hypromellose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508®(T-1508) (BASF Wyandotte Corporation), Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-lOG® or Surfactant 10-G® (Olin Chemicals, Stamford, Conn.); Crodestas SL-40® (Croda, Inc.); and SA9OHCO, which is C₁₈H₃₇CH₂(CON(CH₃)-CH₂(CHOH)₄(CH₂OH)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-derivatized phospholipid, PEG-derivatized cholesterol, PEG-derivatized cholesterol derivative, PEG-derivatized vitamin A, PEG-derivatized vitamin E, lysozyme, random copolymers of vinyl pyrrolidone and vinyl acetate, and the like.

In one embodiment, the mometasone furoate monohydrate formulation does not comprise benzalkonium chloride.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈)dimethylbenzyl ammonium chloride, N-alkyl (C₁₄-₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric surface stabilizers are any nonpolymeric compound, such benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄^{(+):}

(i) none of R₁-R₄ are CH₃;

(ii) one of R₁-R₄ is CH₃;

(iii) three of R₁-R₄ are CH₃;

(iv) all of R₁-R₄ are CH₃;

(v) two of R₁-R₄ are CH₃, one of R₁-R₄ is C₆H₅CH₂, and one of R₁-R₄ is an alkyl chain of seven carbon atoms or less;

(vi) two of R₁-R₄ are CH₃, one of R₁-R₄ is C₆H₅CH₂, and one of R₁-R₄ is an alkyl chain of nineteen carbon atoms or more;

(vii) two of R₁-R₄ are CH₃ and one of R₁-R₄ is the group C₆H₅(CH₂)ₙ, where n>1;

(viii) two of R₁-R₄ are CH₃, one of R₁-R₄ is C₆H₅CH₂, and one of R₁-R₄ comprises at least one heteroatom;

(ix) two of R₁-R₄ are CH₃, one of R₁-R₄ is C₆H₅CH₂, and one of R₁-R₄ comprises at least one halogen;

(x) two of R₁-R₄ are CH₃, one of R₁-R₄ is C₆H₅CH₂, and one of R₁-R₄ comprises at least one cyclic fragment;

(xi) two of R₁-R₄ are CH₃ and one of R₁-R₄ is a phenyl ring; or

(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride(Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HC1, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The surface stabilizers described herein are commercially available and/or can be prepared by techniques known in the art. Most of the surface stabilizers are described in detail in the Handbook of Pharmaceutical Excipients, published jointly by the American Pharmaceutical Association and The Pharmaceutical Society of Great Britain (The Pharmaceutical Press, 2000), specifically incorporated by reference.

3. Other Pharmaceutical Excipients

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride. In one embodiment, the mometasone furoate monohydrate formulation does not comprise benzalkonium chloride as a preservative.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

4. Mometasone Furoate Particle Size

The compositions of the invention comprise mometasone furoate particles which preferably have an effective average particle size of less than about 2000 nm (i.e., 2 microns), less than about 1900 nm, less than less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, or less than about 50 nm, as measured by light-scattering methods, microscopy, or other appropriate methods.

If the nanoparticulate mometasone furoate composition additionally comprises one or more non- mometasone furoate nanoparticulate active agents, then such active agents have an effective average particle size of less than about 2000 nm (i.e., 2 microns). In other embodiments of the invention, the nanoparticulate non- mometasone furoate active agents can have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by the above-noted techniques.

By “an effective average particle size of less than about 2000 nm” it is meant that at least 50% of the nanoparticulate mometasone furoate particles or nanoparticulate non- mometasone furoate active agent particles have a particle size of less than about 2000 nm, when measured by the above-noted techniques. In other embodiments of the invention, at least about 70%, at least about 80%, at least about 90%, at least about 95%, at least about 99%, or at least about 99.9% of the nanoparticulate mometasone furoate particles or nanoparticulate non- mometasone furoate active agent particles have a particle size of less than the effective average, by weight, i.e., less than about 2000 nm, less than about 1900 nm, less than less than about 1800 nm, less than about 1700 nm, etc.

If the nanoparticulate mometasone furoate composition is combined with a conventional or microparticulate mometasone furoate composition or non- mometasone furoate active agent composition, then such a composition is either solubilized or has an effective average particle size of greater than about 2 microns. By “an effective average particle size of greater than about 2 microns” it is meant that at least 50% of the conventional mometasone furoate or conventional non-mometasone furoate active agent particles have a particle size of greater than about 2 microns, by weight, when measured by the above-noted techniques. In other embodiments of the invention, at least about 70%, at least about 80%, at least about 90%, at least about 95%, at least about 99%, or at least about 99.9%, by weight, of the conventional mometasone furoate or conventional non- mometasone furoate active agent particles have a particle size greater than about 2 microns.

In the present invention, the value for D50 of a nanoparticulate mometasone furoate composition is the particle size below which 50% of the mometasone furoate particles fall, by weight. Similarly, D90 is the particle size below which 90% of the mometasone furoate particles fall, by weight.

5. Concentration of Mometasone Furoate and Surface Stabilizers

The relative amounts of mometasone furoate and one or more surface stabilizers can vary widely. The optimal amount of the individual components can depend, for example, upon the hydrophilic lipophilic balance (HLB), melting point, and the surface tension of water solutions of the surface stabilizer, etc.

The concentration of mometasone furoate can vary from about 99.5% to about 0.001%, from about 95% to about 0.1%, or from about 90% to about 0.5%, by weight, based on the total combined dry weight of the mometasone furoate and at least one surface stabilizer, not including other excipients.

The concentration of the at least one surface stabilizer can vary from about 0.5% to about 99.999%, from about 5.0% to about 99.9%, or from about 10% to about 99.5%, by weight, based on the total combined dry weight of the mometasone furoate and at least one surface stabilizer, not including other excipients.

### E. Methods of Making Nanoparticulate Mometasone Furoate Formulations

The mometasone furoate compositions of the invention can be made using, for example, milling, homogenization, precipitation, freezing, template emulsion techniques, supercritical fluid techniques, nano-electrospray techniques, or any combination thereof. Exemplary methods of making nanoparticulate compositions are described in the '684 patent. Methods of making nanoparticulate compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated by reference.

The resultant nanoparticulate mometasone furoate compositions can be utilized in solid, semi-solid, or liquid dosage formulations, such as controlled release formulations, solid dose fast melt formulations, aerosol formulations, nasal formulations, lyophilized formulations, tablets, capsules, solid lozenge, powders, creams, ointments, etc. In a preferred embodiment, the mometasone furoate compositions of the present invention are prepared as a nasal formulation.

1. Milling to Obtain Nanoparticulate Mometasone Furoate Dispersions

Milling mometasone furoate to obtain a nanoparticulate mometasone furoate dispersion comprises dispersing mometasone furoate particles in a liquid dispersion medium in which mometasone furoate is poorly soluble, followed by applying mechanical means in the presence of grinding media, which is preferably less than about 500 micrometers in size, to reduce the particle size of mometasone furoate to the desired effective average particle size. The dispersion media can be any media in which mometasone furoate is poorly soluble, for example, water, safflower oil, ethanol, t-butanol, glycerin, polyethylene glycol (PEG), hexane, or glycol. Water is a preferred dispersion media.

The mometasone furoate particles can be reduced in size in the presence of at least one surface stabilizer. Alternatively, the mometasone furoate particles can be contacted with one or more surface stabilizers after attrition. Other compounds, such as a diluent, can be added to the mometasone furoate /surface stabilizer composition during the size reduction process. Dispersions can be manufactured continuously or in a batch mode.

2. Precipitation to Obtain Nanoparticulate Mometasone Furoate Compositions

Another method of forming the desired nanoparticulate mometasone furoate composition is by microprecipitation. This is a method of preparing stable dispersions of poorly soluble active agents in the presence of one or more surface stabilizers and one or more colloid stability enhancing surface active agents free of any trace toxic solvents or solubilized heavy metal impurities. Such a method comprises, for example: (1) dissolving mometasone furoate in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means.

3. Homogenization to Obtain Nanoparticulate Mometasone Furoate Compositions

Exemplary homogenization methods of preparing nanoparticulate active agent compositions are described in U.S. Pat. No. 5,510,118, for “Process of Preparing Therapeutic Compositions Containing Nanoparticles.”

Such a method comprises dispersing mometasone furoate particles in a liquid dispersion medium in which mometasone furoate is poorly soluble, followed by subjecting the dispersion to homogenization to reduce the particle size of the mometasone furoate to the desired effective average particle size. The mometasone furoate particles can be reduced in size in the presence of at least one surface stabilizer. Alternatively, the mometasone furoate particles can be contacted with one or more surface stabilizers either before or after attrition. Other compounds, such as a diluent, can be added to the mometasone furoate /surface stabilizer composition either before, during, or after the size reduction process. Dispersions can be manufactured continuously or in a batch mode.

### 4. Cryogenic Methodologies to Obtain Nanoparticulate Mometasone Furoate Compositions

Another method of forming the desired nanoparticulate mometasone furoate compositions is by spray freezing into liquid (“SFL”). This technology comprises an organic or organoaqueous solution of mometasone furoate with stabilizers, which is injected into a cryogenic liquid, such as liquid nitrogen. The droplets of the mometasone furoate solution freeze at a rate sufficient to minimize crystallization and particle growth, thus formulating nanostructured mometasone furoate particles. Depending on the choice of solvent system and processing conditions, the nanoparticulate mometasone furoate particles can have varying particle morphology. In the isolation step, the nitrogen and solvent are removed under conditions that avoid agglomeration or ripening of the mometasone furoate particles.

As a complementary technology to SFL, ultra rapid freezing (“URF”) may also be used to created equivalent nanostructured mometasone furoate particles with greatly enhanced surface area. URF comprises an organic or organoaqueous solution of mometasone furoate with stabilizers onto a cryogenic substrate.

### 5. Emulsion Methodologies to Obtain Nanoparticulate Mometasone Furoate Compositions

Another method of forming the desired nanoparticulate mometasone furoate, or a salt or derivative thereof, composition is by template emulsion. Template emulsion creates nanostructured mometasone furoate particles with controlled particle size distribution and rapid dissolution performance. The method comprises an oil-in-water emulsion that is prepared, then swelled with a non-aqueous solution comprising the mometasone furoate and stabilizers. The particle size distribution of the mometasone furoate particles is a direct result of the size of the emulsion droplets prior to loading with the mometasone furoate a property which can be controlled and optimized in this process. Furthermore, through selected use of solvents and stabilizers, emulsion stability is achieved with no or suppressed Ostwald ripening. Subsequently, the solvent and water are removed, and the stabilized nanostructured mometasone furoate particles are recovered. Various mometasone furoate particles morphologies can be achieved by appropriate control of processing conditions.

6. Supercritical Fluid Techniques Used to Obtain Nanoparticulate Mometasone Furoate Compositions

Published International Patent Application No. WO 97/14407 to Pace et al., published Apr. 24, 1997, discloses particles of water insoluble biologically active compounds with an average size of 100 nm to 300 nm that are prepared by dissolving the compound in a solution and then spraying the solution into compressed gas, liquid or supercritical fluid in the presence of appropriate surface modifiers.

7. Nano-Electrospray Techniques Used to Obtain Nanoparticulate Mometasone Furoate Compositions

In electrospray ionization a liquid is pushed through a very small charged, usually metal, capillary. This liquid contains the desired substance, e.g., mometasone furoate (or “analyte”), dissolved in a large amount of solvent, which is usually much more volatile than the analyte. Volatile acids, bases or buffers are often added to this solution as well. The analyte exists as an ion in solution either in a protonated form or as an anion. As like charges repel, the liquid pushes itself out of the capillary and forms a mist or an aerosol of small droplets about 10 μm across. This jet of aerosol droplets is at least partially produced by a process involving the formation of a Taylor cone and a jet from the tip of this cone. A neutral carrier gas, such as nitrogen gas, is sometimes used to help nebulize the liquid and to help evaporate the neutral solvent in the small droplets. As the small droplets evaporate, suspended in the air, the charged analyte molecules are forced closer together. The drops become unstable as the similarly charged molecules come closer together and the droplets once again break up. This is referred to as Coulombic fission because it is the repulsive Coulombic forces between charged analyte molecules that drive it. This process repeats itself until the analyte is free of solvent and is a lone ion.

In nanotechnology the electrospray method may be employed to deposit single particles on surfaces, e.g., particles of mometasone furoate. This is accomplished by spraying colloids and making sure that on average there is not more than one particle per droplet. Consequent drying of the surrounding solvent results in an aerosol stream of single particles of the desired type. Here the ionizing property of the process is not crucial for the application but may be put to use in electrostatic precipitation of the particles.

### F. Methods of Treatment Using the Mometasone Furoate Compositions of the Invention

The present invention is directed to methods of treating a subject in need using the mometasone furoate compositions of the invention. For example, a “subject in need” would include a subject suffering from inflammatory diseases of the airway passages and/or lungs, or a subject afflicted with allergic diseases such as seasonal allergic rhinitis and perennial allergic rhinitis.

As used herein, the term “subject” is used to mean an animal, preferably a mammal, including a human or non-human. The terms patient and subject may be used interchangeably. In addition, the compositions of the present invention can be used for both prophylaxis and treatment of symptoms.

1. Methods and Mometasone Furoate Dosage Forms of the Invention

The mometasone furoate compositions of the invention can be administered to a subject via any conventional means, such as orally or by nasal spray.

If the mometasone furoate compositions are formulated for aerosol inhalation, any suitable device can be used for administration of such a dosage form. Such devices are well known in the art.

The mometasone furoate compositions of the present invention may also contain adjuvants such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

Solid dosage forms for oral administration include, but are not limited to, gels, powders, capsules, tablets, pills, and granules. In such solid dosage forms, the active agent is usually admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable aerosols, emulsions, solutions, suspensions, syrups, and elixirs. In addition to the active agent, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

2. Mometasone Furoate Dosages

The method of the invention comprises administering to a subject an effective amount of a composition comprising mometasone furoate. Depending on the mode of administration, the mometasone furoate compositions of the invention are useful in treating any of the disorders mentioned herein.

‘Therapeutically effective amount’ as used herein with respect to a mometasone furoate dosage, shall mean that dosage that provides the specific pharmacological response for which the drug is administered in a significant number of subjects in need of such treatment. It is emphasized that ‘therapeutically effective amount,’ administered to a particular subject in a particular instance will not always be effective in prophylaxis or treatment of the diseases described herein, even though such dosage is deemed a ‘therapeutically effective amount’ by those skilled in the art. It is to be further understood that mometasone furoate dosages are, in particular instances, measured as oral dosages, or with reference to drug levels as measured in blood.

One of ordinary skill will appreciate that effective amounts of mometasone furoate can be determined empirically and can be employed in pure form or, where such forms exist, in pharmaceutically acceptable salt, ester, or prodrug form. Actual dosage levels of mometasone furoate in the compositions of the invention may be varied to obtain an amount of mometasone furoate that is effective to obtain a desired therapeutic response for a particular composition and method of administration. The selected dosage level therefore depends upon the desired therapeutic effect, the route of administration, the potency of the administered mometasone furoate, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

3. Exemplary Disorders That Can be Treated with the Mometasone Furoate Compositions of the Invention

The mometasone furoate compositions can be used for treating disorders such as respiratory related diseases or conditions. In particular, treatment of diseases of airway passages and lungs in accordance with the invention may be symptomatic or prophylactic treatment. Diseases of airway passages and lungs can be considered inflammatory airways diseases to which the present invention is applicable and include asthma of whatever type or genesis, including both intrinsic (non-allergic) asthma and extrinsic (allergic) asthma. Treatment of asthma is also to be understood as embracing treatment of subjects, e.g. of less than 4 or 5 years of age, exhibiting wheezing symptoms and diagnosed or diagnosable as “wheezy infants,” an established patient category of major medical concern and now often identified as incipient or early-phase asthmatics (for convenience this particular asthmatic condition is referred to as “wheezy-infant syndrome”).

The term “asthma” as used herein includes any asthmatic condition marked by recurrent attacks of paroxysmal dyspnea (i.e., reversible obstructive airway passage disease) with wheezing due to spasmodic contraction of the bronchi. Asthmatic conditions which may be treated or prevented in accordance with this invention include allergic asthma and bronchial allergy characterized by manifestations in sensitized persons provoked by a variety of factors including exercise, especially vigorous exercise (exercise induced bronchospasm), irritant particles (e.g., pollen, dust, cotton, dander, etc.), as well as mild to moderate asthma, chronic asthma, severe chronic asthma, severe and unstable asthma, nocturnal asthma, and psychological stresses.

Prophylactic efficacy in the treatment of asthma will be evidenced by reduced frequency or severity of symptomatic attack, e.g. of acute asthmatic or bronchoconstrictor attack, improvement in lung function, or improved airways hyperreactivity. It may further be evidenced by a reduced requirement for other, symptomatic therapy, i.e., therapy for or intended to restrict or abort symptomatic attack when it occurs, for example, anti-inflammatory (e.g., corticosteroid) or bronchodilatory.

Prophylactic benefit in asthma may in particular be apparent in subjects prone to “morning dipping.” “Morning dipping” is a recognized asthmatic syndrome common to a substantial percentage of asthmatics and characterized by asthma attack, e.g., between the hours of about 4 to 6 am, i.e., at a time normally substantially distant from any previously administered symptomatic asthma therapy.

The compositions of the present invention are also suitable for treating other diseases of airway passages, such as seasonal (e.g., hay fever) or perennial rhinitis, which are characterized by seasonal or perennial sneezing, rhinorrhea, nasal congestion, pruritis and eye itching, redness and tearing, and nonallergic (vasomotor) rhinitis (i.e., eosinophilic nonallergic rhinitis which is found in patients with negative skin tests and those who have numerous eosinophils in their nasal secretions). The term “allergic rhinitis” as used herein includes any allergic reaction of the nasal mucosa.

Other inflammatory or obstructive airways diseases and conditions to which the present invention is applicable include acute lung injury (ALI), acute respiratory distress syndrome (ARDS), chronic obstructive pulmonary, airways or lung disease (COPD, COAD, or COLD), including chronic bronchitis and emphysema, bronchiectasis, and exacerbation of airways hyperreactivity consequent to other drug therapy, in particular other inhaled drug therapy. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis byssinosis, inflammatory bowel diseases, including Crohn's disease and ulcerative colitis, Whipple's disease, AIDS related pneumonia, and skin conditions treatable with topical corticosteroids.

The following examples are given to illustrate the present invention. It should be understood, however, that the invention is not to be limited to the specific conditions or details described in this example. Throughout the specification, any and all references to a publicly available document, including a U.S. patent, are specifically incorporated by reference.

### Example 1

The purpose of this example is to prepare a nanoparticulate dispersion of mometasone furoate.

A mixture of 5% w/w mometasone furoate and 2.5% of an ionic surface stabilizer in saline is milled for 1.25 hours under high energy milling conditions in a DYNO®-Mill KDL (Willy A. Bachofen AG, Maschinenfabrik, Basel, Switzerland) equipped with a 150 cc batch chamber. 200 μm polymeric attrition media (The Dow Chemical Co., Midland, Mich.) is utilized in the milling process.

Particle size analysis of the milled mometasone furoate composition is conducted using a Horiba LA-910 particle size analyzer (Irvine, Calif.), showing a final mometasone furoate average particle size of 92 nm.

The composition is stable for at least 8 weeks at 5° C., 25° C., and 40° C.

### Example 2

The purpose of this example is to prepare a sterile filtered nanoparticulate mometasone furoate composition.

The milled mometasone furoate composition of Example 1 is successfully sterile filtered using 0.8/0.2 micron syringe filters. The sterile filtered composition is stable for at least 8 weeks at 5° C., 25° C., and 40° C.

### Example 3

The purpose of this example is to prepare a nanoparticulate dispersion of mometasone furoate.

A mixture of 5% w/w mometasone furoate and 2.5% of a cationic surface stabilizer in saline is milled for 1.25 hours under high energy milling conditions in a DYNO®-Mill KDL (Willy A. Bachofen AG, Maschinenfabrik, Basel, Switzerland) equipped with a 150 cc batch chamber. 200 p.m polymeric attrition media (The Dow Chemical Co., Midland, Mich.) is utilized in the milling process.

Particle size analysis of the milled mometasone furoate composition is conducted using a Horiba LA-910 particle size analyzer (Irvine, Calif.), showing a final mometasone furoate average particle size of 92 nm.

The composition is stable for at least 8 weeks at 5° C., 25° C., and 40° C.

### Example 4

The purpose of this example is to prepare a nanoparticulate dispersion of mometasone furoate.

A mixture of 5% w/w mometasone furoate and 2.5% of a non-ionic surface stabilizer in saline is milled for 1.25 hours under high energy milling conditions in a DYNO®-Mill KDL (Willy A. Bachofen AG, Maschinenfabrik, Basel, Switzerland) equipped with a 150 cc batch chamber. 200 μm polymeric attrition media (The Dow Chemical Co., Midland, Mich.) is utilized in the milling process.

Particle size analysis of the milled mometasone furoate composition is conducted using a Horiba LA-910 particle size analyzer (Irvine, Calif.), showing a final mometasone furoate average particle size of 92 nm.

The composition is stable for at least 8 weeks at 5° C., 25° C., and 40° C.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

1. A mometasone furoate composition comprising:
(a) particles of mometasone furoate, wherein at least 50% of the mometasone furoate particles have a particle size of less than 1000 nm; and
(b) at least one surface stabilizer adsorbed on the surface of the mometasone furoate particles, and
(c) a liquid dispersion medium,
wherein the mometasone furoate particles exist in a phase selected from the group consisting of a crystalline phase, an amorphous phase, and a semi-crystalline phase.

2. The composition of claim 1, wherein at least 50% of the mometasone furoate particles have a particle size selected from the group consisting of less than 900 nm, less than 800 nm, less than 700 nm, less than 600 nm, less than 500 nm, less than 400 nm, less than 300 nm, less than 250 nm, less than 200 nm, less than 100 nm, less than 75 nm, and less than 50 nm.

3.-4. (canceled)

5. The composition of claim 1, wherein the composition is formulated:
(a) for administration selected from the group consisting of parenteral, oral, pulmonary, intravenous, rectal, ophthalmic, colonic, intracisternal, intravaginal, intraperitoneal, ocular, otic, local, buccal, nasal, bioadhesive and topical administration;
(b) into a dosage form selected from the group consisting of liquid dispersions, gels, aerosols, ointments, and creams; or (c) any combination of (a) and (b).

6. The composition of claim 1 further comprising one or more phaimaceutically acceptable excipients, carriers, or a combination thereof

7. The composition of claim 1, wherein
(a) the amount of mometasone furoate is selected from the group consisting of from about 99.5% to about 0.001%, from about 95% to about 0.1%, and from about 90% to about 0.5%, by weight, based on the total combined weight of mometasone furoate and at least one surface stabilizer, not including other excipients;
(b) at least one surface stabilizer is present in an amount selected from the group consisting of from about 0.5% to about 99.999% by weight, from about 5.0% to about 99.9% by weight, and from about 10% to about 99.5% by weight, based on the total combined dry weight of mometasone furoate and at least one surface stabilizer, not including other excipients; or (c) a combination of (a) and (b).

8. The composition of claim 1, comprising at least two surface stabilizers.

9. The composition of claim 1, wherein the surface stabilizer is selected from the group consisting of a non-ionic surface stabilizer, an ionic surface stabilizer, an anionic surface stabilizer, a cationic surface stabilizer, and a zwitterionic surface stabilizer.

10. The composition of claim 9, wherein the at least one surface stabilizer is selected from the group consisting of albumin, human serum albumin, bovine albumin, cetyl pyridinium chloride, gelatin, casein, phosphatides, dextran, glycerol, gum acacia, cholesterol, tragacanth, stearic acid, calcium stearate, glycerol monostearate, cetostearyl alcohol, cetomacrogol emulsifying wax, sorbitan esters, polyoxyethylene alkyl ethers, polyoxyethylene castor oil, polyoxyethylene sorbitan fatty acid esters, polyethylene glycols, dodecyl trimethyl ammonium bromide, polyoxyethylene stearates, colloidal silicon dioxide, phosphates, sodium dodecyl sulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-derivatized phospholipid, PEG-derivatized cholesterol, PEG-derivatized cholesterol, PEG-derivatized vitamin A, PEG-derivatized vitamin E, random copolymers of vinyl acetate and vinyl pyrrolidone, a cationic polymer, a cationic biopolymer, a cationic polysaccharide, a cationic cellulosic, a cationic alginate, a cationic nonpolymeric compound, a cationic phospholipid, poly-n-methylpyridinium, anthryul pyridinium chloride, chitosan, polylysine, polyvinylimidazole, polybrene, polymethylmethacrylate trimethylammoniumbromide bromide (PMMTMABr), hexyldesyltrimethylammonium bromide (HDMAB), polyvinylpyrrolidone-2-dimethylaminoethyl methacrylate dimethyl sulfate, 1,2 Dipalmitoyl-sn-Glycero-3-Phosphoethanolamine-N-[Amino(Polyethylene Glycol)2000] (sodium salt), Poly(2-methacryloxyethyl trimethylammonium bromide), poloxamines, lysozyme, alginic acid, carrageenan, nonionic poly (ethylene oxide) polymers, cationic lipids, sulfonium, phosphonium, quarternary ammonium compounds, stearyltrimethylammonium chloride, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium bromide, C₁₂₋₁₅dimethyl hydroxyethyl ammonium chloride, C₁₂₋₁₅ammonium bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium hal ogenides, tricetyl methyl ammonium chloride, decyltrimethylammonium bromide, dodecyltriethylammonium bromide, tetradecyltrimethylammonium bromide, methyl trioctylammonium chloride, polyquaternium 10, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, quaternized ammonium salt polymers, alkyl pyridinium salts, amines, protonated quaternary acrylamides, methylated quaternary polymers, cationic guar, alkylamines, dialkylamines, alkanolamines, polyethylencpolyamines, N,N-dialkylaminoalkyl acrylates, vinyl pyridine, amine salts, lauryl amine acetate, stearyl amine acetate, alkylpyridinium salt, alkylimidazolium salt, amine oxides, imide azolinium salts, benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quatemium-15), distearyldimonium chloride (Quatemium-5), dodecyl dimethyl ethylbenzyl ammonium chloride(Quatemium-14), Quaternium-22, Quaternium-26, Quatemium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquatemium-1, procainehydrochloride, cocobetaine, stearalkonium bentonite, stearalkoniumhectonite, stearyl trihydroxyethyl propylenediamine dihydrofluoride, tall owtrimonium chloride, hexadecyltrimethyl ammonium bromide, and combinations thereof.

11. The composition according to claim 9, wherein the composition is bioadhesive.

12. The composition of claim 1, wherein the phai iacokinetic profile of the composition is not significantly affected by the fed or fasted state of a subject ingesting the composition.

13. The composition of claim 1, wherein the composition does not produce significantly different absorption levels when administered under fed as compared to fasting conditions.

14. The composition of claim 13, wherein the difference in absorption of the mometasone furoate composition of the invention, when administered in the fed versus the fasted state, is selected from the group consisting of less than 100%, less than 90%, less than 80%, less than 70%, less than 60%, less than 50%, less than 40%, less than 30%, less than 25%, less than 20%, less than 15%, less than 10%, less than 5%, and less than 3%.

15. The composition of claim 1, wherein administration of the composition to a subject in a fasted state is bioequivalent to administration of the composition to a subject in a fed state.

16. The composition of claim 15, wherein “bioequivalency” is established by:
(a) a 90% Confidence Interval of between 0.80 and 1.25 for both Cₘₐₓ and AUC; or
(b) a 90% Confidence Interval of between 0.80 and 1.25 for AUC and a 90% Confidence Interval of between 0.70 to 1.43 for Cₘₐₓ.

17. The composition of claim 1, wherein:
(a) the Tₘₐₓ of the mometasone furoate, when assayed in the plasma of a mammalian subject following administration, is less than the Tₘₐₓ for a non-nanoparticulate composition of the same mometasone furoate, administered at the same dosage;
(b) the Cₘₐₓ of the mometasone furoate, when assayed in the plasma of a mammalian subject following administration, is greater than the Cₘₐₓ for a non-nanoparticulate composition of the same mometasone furoate, administered at the same dosage;
(c) the AUC of the mometasone furoate, when assayed in the plasma of a mammalian subject following administration, is greater than the AUC for a non-nanoparticulate composition of the same mometasone furoate, administered at the same dosage; or
(d) any combination of (a), (b), and (c).

18. The composition of claim 17, wherein:
(a) the Tₘₐₓ is selected from the group consisting of not greater than 90%, not greater than 80%, not greater than 70%, not greater than 60%, not greater than 50%, not greater than 30%, not greater than 25%, not greater than 20%, not greater than 15%, not greater than 10%, and not greater than 5% of the Tₘₐₓ exhibited by a non-nanoparticulate composition of the same mometasone furoate, administered at the same dosage;
(b) the C. is selected from the group consisting of at least 50%, at least 100%, at least 200%, at least 300%, at least 400%, at least 500%, at least 600%, at least 700%, at least 800%, at least 900%, at least 1000%, at least 1100%, at least 1200%, at least 1300%, at least 1400%, at least 1500%, at least 1600%, at least 1700%, at least 1800%, or at least 1900% greater than the Cₘₐₓ exhibited by a non-nanoparticulate composition of the same mometasone furoate, administered at the same dosage;
(c) the AUC is selected from the group consisting of at least 25%, at least 50%, at least 75%, at least 100%, at least 125%, at least 150%, at least 175%, at least 200%, at least 225%, at least 250%, at least 275%, at least 300%, at least 350%, at least 400%, at least 450%, at least 500%, at least 550%, at least 600%, at least 750%, at least 700%, at least 750%, at least 800%, at least 850%, at least 900%, at least 950%, at least 1000%, at least 1050%, at least 1100%, at least 1150%, or at least 1200% greater than the AUC exhibited by the non-nanoparticulate formulation of the same mometasone furoate, administered at the same dosage; or
(d) any combination of (a), (b), and (c).

19. A mometasone furoate composition comprising:
(a) particles of mometasone furoate or a salt thereof, wherein at least 50% of the mometasone furoate particles have a particle size of less than 200 nm; and
(b) at least one surface stabilizer adsorbed on the surface of the mometasone furoate particles, and
(c) a liquid dispersion medium,
wherein the composition is capable of being sterilized by filtration and wherein the mometasone furoate particles exist in a phase selected from the group consisting of a crystalline phase, an amorphous phase, and a semi-crystalline phase.

20. (canceled)

21. The mometasone furoate composition of claim 19, comprising at least two surface stabilizers.

22. The mometasone furoate composition of claim 19, wherein at least 99.9% of the mometasone furoate particles have a particle size of less than 200 nm.

23. The mometasone furoate composition of claim 19, wherein at least 90% of the mometasone furoate particles have a particle size of less than 130 nm.

24. (canceled)

25. The nanoparticulate mometasone furoate composition of claim 19, wherein at least 50% of the mometasone furoate particles have a particle size of less than 150 nm.

26. The composition of claim 25, wherein at least 50% of the mometasone furoate particles have a particle size selected from the group consisting of less than 140 nm, less than 130 nm, less than 120 nm, less than 110 nm, less than 100 nm, less than 90 nm, less than 80 nm, less than 70 nm, less than 60 nm, and less than 50 nm.

27.-28. (canceled)

29. The composition of claim 25, wherein the composition is formulated:
(a) for administration selected from the group consisting of parenteral, oral, pulmonary, intravenous, rectal, ophthalmic, colonic, intracisternal, intravaginal, intraperitoneal, ocular, otic, local, buccal, nasal, bioadhesive and topical administration;
(b) into a dosage form selected from the group consisting of liquid dispersions, gels, aerosols, ointments, and creams; or
(c) any combination of (a) and (b).

30. The composition of claim 25 further comprising one or more pharmaceutically acceptable excipients, carriers, or a combination thereof.

31. The composition of claim 25, wherein:
(a) the mometasone furoate is present in an amount selected from the group consisting of from about 99.5% to about 0.001%, from about 95% to about 0.1%, and from about 90% to about 0.5%, by weight, based on the total combined dry weight of the mometasone furoate and at least one surface stabilizer, not including other excipients;
(b) the at least one surface stabilizer is present in an amount selected from the group consisting of from about 0.5% to about 99.999%, from about 5.0% to about 99.9%, and from about 10% to about 99.5%, by weight, based on the total combined dry weight of the mometasone furoate and at least one surface stabilizer, not including other excipients; or
(c) a combination of (a) and (b).

32. The composition of claim 25, comprising at least two surface stabilizers.

33. The composition of claim 25, wherein the surface stabilizer is selected from the group consisting of a non-ionic surface stabilizer, an ionic surface stabilizer, an anionic surface stabilizer, a cationic surface stabilizer, and a zwitterionic surface stabilizer.

34. The composition of claim 33, wherein the at least one surface stabilizer is selected from the group consisting of albumin, human serum albumin, bovine albumin, cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl P-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-derivatized phospholipid, PEG-derivatized cholesterol, PEG-derivatized cholesterol, PEG-derivatized vitamin A, PEG-derivatized vitamin E, random copolymers of vinyl acetate and vinyl pyrrolidone, a cationic polymer, a cationic biopolymer, a cationic polysaccharide, a cationic cellulosic, a cationic alginate, a cationic nonpolymeric compound, a cationic phospholipid, poly-n-methylpyridinium, anthryul pyridinium chloride, chitosan, polylysine, polyvinylimidazole, polybrene, polymethylmethacrylate trimethylammoniumbromide bromide (PMMTMABr), hexyldesyltrimethylammonium bromide (HDMAB), polyvinylpyrrolidone-2-dimethylaminoethyl methacrylate dimethyl sulfate, 1,2 Dipalmitoyl-sn-Glycero-3-Phosphoethanolamine-N-[Amino(Polyethylene Glycol)2000] (sodium salt), Poly(2-methacryloxyethyl trimethylammonium bromide), poloxamines, lysozyme, alginic acid, carrageenan, nonionic poly (ethylene oxide) polymers, cationic lipids, sulfonium, phosphonium, quarternary ammonium compounds, stearyltrimethylammonium chloride, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium bromide, C₁₂₋₁₅dimethyl hydroxyethyl ammonium chloride, C₁₂₋₁₅dimethyl hydroxyethyl ammonium bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialyl benzenealkyl ammonium chloride, lauryl trimethyl ammonium chloride, alkylbenzyl methyl ammonium chloride, alkyl benzyl dimethyl ammonium bromide, C₁₂), C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, polyquatemium 10, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, quaternized ammonium salt polymers, alkyl pyridinium salts, amines, protonated quaternary acrylamides, methylated quaternary polymers, cationic guar, alkylamines, dialkylamines, alkanolamines, polyethylenepolyamines, N,N-dialkylaminoalkyl acrylates, vinyl pyridine, amine salts, lauryl amine acetate, stearyl amine acetate, alkylpyridinium salt, alkylimidazolium salt, amine oxides, imide azolinium salts, benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quatemium-15), distearyldimonium chloride (Quatemium-5), dodecyl dimethyl ethylbenzyl ammonium chloride(Quaternium-14), Quaternium-22, Quaternium-26, Quatemium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkomum chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, hexadecyltrimethyl ammonium bromide, and combinations thereof.

35. The composition according to claim 33, wherein the composition is bioadhesive.

36. A method of making a mometasone furoate composition comprising contacting particles of mometasone furoate or a salt thereof with at least one surface stabilizer in the presence of a liquid dispersion medium for a time and under conditions sufficient to provide a mometasone furoate composition in which at least 50% of the mometasone furoate particles have a particle size of less than 1000 nm and the surface stabilizer is adsorbed on the surface of the mometasone furoate particles, wherein the mometasone furoate particles exist in a phase selected from the group consisting of a crystalline phase, an amorphous phase, and a semi-crystalline phase.

37. The method of claim 36, wherein said contacting comprises milling, wet milling, homogenizing, precipitation, freezing, template emulsion techniques, supercritical fluid techniques, nano-electrospray techniques, or any combination thereof.

38. The method of claim 36, wherein at least 50% of the mometasone furoate particles have a particle size selected from the group consisting of less than 900 nm, less than 800 nm, less than 700 nm, less than 600 nm, less than 500 nm, less than 400 nm, less than 300 nm, less than 250 nm, less than 200 nm, less than 150 nm, less than 140 nm, less than 130 nm, less than 120 nm, less than 110 nm, less than 100 nm, less than 90 nm, less than 80 nm, less than 70 nm, less than 60 nm, and less than 50 nm.

39. (canceled)

40. The method of claim 36, further comprising sterilizing the mometasone furoate composition by filtration.

41. A method of treating a subject in need of either symptomatic or prophylactic treatment with a mometasone furoate composition comprising administering to the subject an effective amount of a composition comprising particles of mometasone furoate or a salt thereof, at least one surface stabilizer, and a liquid dispersion medium,
wherein:
(i) at least 50% of the mometasone furoate particles have a particle size of less than 1000 nm,
(ii) the surface stabilizer is adsorbed on the surface of the mometasone furoate particles, and
(iii) a liquid dispersion medium.

42. The method of claim 41, wherein at least 50% of the mometasone furoate particles have a particle size selected from the group consisting of less than 900 nm, less than 800 nm, less than 700 nm, less than 600 nm, less than 500 nm, less than 400 nm, less than 300 nm, less than 250 nm, less than 200 nm, less than 150 nm, less than 140 nm, less than 130 nm, less than 120 nm, less than 110 nm, less than 100 nm, less than 90 nm, less than 80 nm, less than 70 nm, less than 60 nm, and less than 50 nm.

43. (canceled)

44. The method of claim 41, wherein the subject has a condition selected from the group consisting of a respiratory related illness, inflammatory airways diseases, asthma, emphysema, respiratory distress syndrome, seasonal rhinitis, perennial rhinitis, seasonal allergic rhinitis, seasonal nonallergic rhinitis, perennial allergic rhinitis, perennial nonallergic rhinitis, skin conditions treatable with topical corticosteroids, intrinsic (non-allergic) asthma, extrinsic (allergic) asthma, wheezy-infant syndrome, acute lung injury, acute respiratory distress syndrome, chronic obstructive pulmonary disease, chronic obstructive airways disease, chronic obstructive lung disease, chronic bronchitis, emphysema, bronchiectasis, exacerbation of airways hyperreactivity consequent to other drug therapy, and pneumoconiosis.

45. The method of claim 41, wherein the prophylactic efficacy of the treatment is evidenced by one or more characteristics selected from the group consisting of reduced frequency of symptomatic attack, reduced severity of symptomatic attack, improvement in lung function, improved airways hyperreactivity, and a reduced requirement for other symptomatic therapy.

46. The method of claim 41, wherein the subject is a human.

47. The composition of claim 1, wherein at least 70% of the mometasone furoate particles have a particle size selected from the group consisting of less than 900 nm, less than 800 nm, less than 700 nm, less than 600 nm, less than 500 nm, less than 400nm, less than 300 nm, less than 250 nm, less than 200 nm, less than 100 nm, less than 75 nm, and less than 50 nm.

48. The composition of claim 1, wherein at least 80% of the mometasone furoate particles have a particle size selected from the group consisting of less than 900 nm, less than 800 nm, less than 700 nm, less than 600 nm, less than 500 nm, less than 400 nm, less than 300 nm, less than 250 nm, less than 200 nm, less than 100 nm, less than 75 nm, and less than 50 nm.

49. The composition of claim 1, wherein at least 90% of the mometasone furoate particles have a particle size selected from the group consisting of less than 900 nm, less than 800 nm, less than 700 nm, less than 600 nm, less than 500 nm, less than 400 nm, less than 300 nm, less than 250 nm, less than 200 nm, less than 100 nm, less than 75 nm, and less than 50 nm.

50. The composition of claim 1, wherein at least 95% of the mometasone furoate particles have a particle size selected from the group consisting of less than 900 nm, less than 800 nm, less than 700 nm, less than 600 nm, less than 500 nm, less than 400 nm, less than 300 nm, less than 250 nm, less than 200 nm, less than 100 nm, less than 75 nm, and less than 50 nm.

51. The composition of claim 1, wherein at least 99% of the mometasone furoate particles have a particle size selected from the group consisting of less than 900 nm, less than 800 nm, less than 700 nm, less than 600 nm, less than 500 nm, less than 400 nm, less than 300 nm, less than 250 nm, less than 200 nm, less than 100 nm, less than 75 nm, and less than 50 nm.

52. The composition of claim 1, wherein at least 99.9% of the mometasone furoate particles have a particle size selected from the group consisting of less than 900 nm, less than 800 nm, less than 700 nm, less than 600 nm, less than 500 nm, less than 400 nm, less than 300 nm, less than 250 nm, less than 200 nm, less than 100 nm, less than 75 nm, and less than 50 nm.

53. The composition of claim 25, wherein at least 70% of the mometasone furoate particles have a particle size selected from the group consisting of less than 140 nm, less than 130 nm, less than 120 nm, less than 110 nm, less than 100 nm, less than 90 nm, less than 80 nm, less than 70 nm, less than 60 nm, and less than 50 nm.

54. The composition of claim 25, wherein at least 80% of the mometasone furoate particles have a particle size selected from the group consisting of less than 140 nm, less than 130 nm, less than 120 nm, less than 110 nm, less than 100 nm, less than 90 nm, less than 80 nm, less than 70 nm, less than 60 nm, and less than 50 nm.

55. The composition of claim 25, wherein at least 90% of the mometasone furoate particles have a particle size selected from the group consisting of less than 140 nm, less than 130 nm, less than 120 nm, less than 110 nm, less than 100 nm, less than 90 nm, less than 80 nm, less than 70 nm, less than 60 nm, and less than 50 nm.

56. The composition of claim 25, wherein at least 95% of the mometasone furoate particles have a particle size selected from the group consisting of less than 140 nm, less than 130 nm, less than 120 nm, less than 110 nm, less than 100 nm, less than 90 nm, less than 80 nm, less than 70 nm, less than 60 nm, and less than 50 nm.

57. The composition of claim 25, wherein at least 99% of the mometasone furoate particles have a particle size selected from the group consisting of less than 140 nm, less than 130 nm, less than 120 nm, less than 110 nm, less than 100 nm, less than 90 nm, less than 80 nm, less than 70 nm, less than 60 nm, and less than 50 nm.

58. The composition of claim 25, wherein at least 99.9% of the mometasone furoate particles have a particle size selected from the group consisting of less than 140 nm, less than 130 nm, less than 120 nm, less than 110 nm, less than 100 nm, less than 90 nm, less than 80 nm, less than 70 nm, less than 60 nm, and less than 50 nm.

59. The method of claim 36, wherein at least 70% of the mometasone furoate particles have a particle size selected from the group consisting of less than 1900 nm, less than less than 1800 nm, less than 1700 nm, less than 1600 nm, less than 1500 nm, less than 1100 nm, less than 1300 nm, less than 1200 nm, less than 1100 nm, less than 1000 nm, less than 900 nm, less than 800 nm, less than 700 nm, less than 600 nm, less than 500 nm, less than 400 nm, less than 300 nm, less than 250 nm, less than 200 nm, less than 150 nm, less than 140 nm, less than 130 nm, less than 120 nm, less than 110 nm, less than 100 nm, less than 90 nm, less than 80 nm, less than 70 nm, less than 60 nm, and less than 50 nm.

60. The method of claim 36, wherein at least 80% of the mometasone furoate particles have a particle size selected from the group consisting of less than 900 nm, less than 800 nm, less than 700 nm, less than 600 nm, less than 500 nm, less than 400 nm, less than 300 nm, less than 250 nm, less than 200 nm, less than 150 nm, less than 140 nm, less than 130 nm, less than 120 nm, less than 110 nm, less than 100 nm, less than 90 nm, less than 80 nm, less than 70 nm, less than 60 nm, and less than 50 nm.

61. The method of claim 36, wherein at least 90% of the mometasone furoate particles have a particle size selected from the group consisting of less than 900 nm, less than 800 nm, less than 700 nm, less than 600 nm, less than 500 nm, less than 400 nm, less than 300 nm, less than 250 nm, less than 200 nm, less than 150 nm, less than 140 nm, less than 130 nm, less than 120 nm, less than 110 nm, less than 100 nm, less than 90 nm, less than 80 nm, less than 70 nm, less than 60 nm, and less than 50 nm.

62. The method of claim 36, wherein at least 95% of the mometasone furoate particles have a particle size selected from the group consisting of less than 900 nm, less than 800 nm, less than 700 nm, less than 600 nm, less than 500 nm, less than 400 nm, less than 300 nm, less than 250 nm, less than 200 nm, less than 150 nm, less than 140 nm, less than 130 nm, less than 120 nm, less than 110 nm, less than 100 nm, less than 90 nm, less than 80 nm, less than 70 nm, less than 60 nm, and less than 50 nm.

63. The method of claim 36, wherein at least 99% of the mometasone furoate particles have a particle size selected from the group consisting of less than 900 nm, less than 800 nm, less than 700 nm, less than 600 nm, less than 500 nm, less than 400 nm, less than 300 nm, less than 250 nm, less than 200 nm, less than 150 nm, less than 140 nm, less than 130 nm, less than 120 nm, less than 110 nm, less than 100 nm, less than 90 nm, less than 80 nm, less than 70 nm, less than 60 nm, and less than 50 nm.

64. The method of claim 36, wherein at least 99.9% of the mometasone furoate particles have a particle size selected from the group consisting of less than 900 nm, less than 800 nm, less than 700 nm, less than 600 nm, less than 500 nm, less than 400 nm, less than 300 nm, less than 250 nm, less than 200 nm, less than 150 nm, less than 140 nm, less than 130 nm, less than 120 nm, less than 110 nm, less than 100 nm, less than 90 nm, less than 80 nm, less than 70 nm, less than 60 nm, and less than 50 nm.

65. The method of claim 41, wherein at least 70% of the mometasone furoate particles have a particle size selected from the group consisting of less than 900 nm, less than 800 nm, less than 700 nm, less than 600 nm, less than 500 nm, less than 400 nm, less than 300 nm, less than 250 nm, less than 200 nm, less than 150 nm, less than 140 nm, less than 130 nm, less than 120 nm, less than 110 nm, less than 100 nm, less than 90 nm, less than 80 nm, less than 70 nm, less than 60 nm, and less than 50 nm.

66. The method of claim 41, wherein at least 80% of the mometasone furoate particles have a particle size selected from the group consisting of less than 900 nm, less than 800 nm, less than 700 nm, less than 600 nm, less than 500 nm, less than 400 nm, less than 300 nm, less than 250 nm, less than 200 nm, less than 150 nm, less than 140 nm, less than 130 nm, less than 120 nm, less than 110 nm, less than 100 nm, less than 90 nm, less than 80 nm, less than 70 nm, less than 60 nm, and less than 50 nm.

67. The method of claim 41, wherein at least 90% of the mometasone furoate particles have a particle size selected from the group consisting of less than 900 nm, less than 800 nm, less than 700 nm, less than 600 nm, less than 500 nm, less than 400 nm, less than 300 nm, less than 250 nm, less than 200 nm, less than 150 nm, less than 140 nm, less than 130 nm, less than 120 nm, less than 110 nm, less than 100 nm, less than 90 nm, less than 80 nm, less than 70 nm, less than 60 nm, and less than 50 nm.

68. The method of claim 41, wherein at least 95% of the mometasone furoate particles have a particle size selected from the group consisting of less than 900 nm, less than 800 nm, less than 700 nm, less than 600 nm, less than 500 nm, less than 400 nm, less than 300 nm, less than 250 nm, less than 200 nm, less than 150 nm, less than 140 nm, less than 130 nm, less than 120 nm, less than 110 nm, less than 100 nm, less than 90 nm, less than 80 nm, less than 70 nm, less than 60 nm, and less than 50 nm.

69. The method of claim 41, wherein at least 99% of the mometasone furoate particles have a particle size selected from the group consisting of less than 900 nm, less than 800 nm, less than 700 nm, less than 600 nm, less than 500 nm, less than 400 nm, less than 300 nm, less than 250 nm, less than 200 nm, less than 150 nm, less than 140 nm, less than 130 nm, less than 120 nm, less than 110 nm, less than 100 nm, less than 90 nm, less than 80 nm, less than 70 nm, less than 60 nm, and less than 50 nm.

70. The method of claim 41, wherein at least 99.9% of the mometasone furoate particles have a particle size selected from the group consisting of less than less than 900 nm, less than 800 nm, less than 700 nm, less than 600 nm, less than 500 nm, less than 400 nm, less than 300 nm, less than 250 nm, less than 200 nm, less than 150 nm, less than 140 nm, less than 130 nm, less than 120 nm, less than 110 nm, less than 100 nm, less than 90 nm, less than 80 nm, less than 70 nm, less than 60 nm, and less than 50 nm.
